(19) United States
(12) Patent Application Publication
(10) Pub. No.: US 2004/0152727 A1
(43) Pub. Date: Aug. 5, 2004

(54) Novel use

(76) Inventors: Douglas William Pierre Hay, King of Prussia, PA (US); Gareth John Sanger, Essex (GB); Tadataka Yamada, King of Prussia, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/478,272

(22) PCT Filed: May 17, 2002
(86) PCT No.: PCT/US02/15911

(30) Foreign Application Priority Data
May 18, 2001 (GB) 0112208.4
Dec. 6, 2001 (GB) 0129268.9

(51) Int. Cl. A61K 31/4709; A61K 31/47

(52) U.S. Cl. 514/311; 514/314

(57) ABSTRACT

A method for the treatment and/or prophylaxis of conditions characterised by altered bowel function and/or visceral pain in humans or non-human mammals, which method comprises the administration of an effective, non-toxic and pharmaceutically acceptable amount of an NK3 receptor antagonist, wherein the condition characterised by altered bowel function and/or visceral pain is selected from certain irritable bowel syndrome conditions, functional abdominal bloating, functional constipation, functional diarrhea, other bowel conditions and functional abdominal pain.

This invention relates to a novel use, in particular to a use in a method for the treatment and/or prophylaxis of conditions characterised by altered bowel function and/or visceral pain.

The mammalian peptide Neurokinin B (NKB) belongs to the Tachykinin (TK) peptide family which also include Substance P (SP) and Neurokinin A (NKA). Pharmacological and molecular biological evidence has shown the existence of three subtypes of TK receptor (NK₁, NK₂ and NK₃) and NKB binds preferentially to the NK₃ receptor although this and the other tachykinins are relatively promiscuous in terms of their abilities to recognise each of the NK receptors (Maggi et al, 1993, J. Auton. Pharmacol., 13, 23-93).

Selective peptidic NK₃ receptor antagonists are known (Drapeau, 1990 Regul. Pept., 31, 125-135), and findings with peptidic NK₃ receptor agonists suggest that NKB, by activating the NK₃ receptor, has a key role in the modulation of neural input in airways, skin, spinal cord and nigro-striatal pathways (Myers and Undem, 1993, J.Phisiol., 470, 665-679; Counture et al., 1993, Regul. Peptides, 46,426-429; Mccarson and Krause, 1994, J. Neurosci., 14 (2), 712-720; Arenas et al. 1991, J.Neurosci., 11, 2332-8).

The Rome diagnostic criteria (see Gut, 1999; 45 (Suppl. II) II43 -II47) recognise that functional bowel disorder includes the following distinct groups: irritable bowel syndrome (IBS), functional abdominal bloating, functional constipation and functional diarrhea. IBS is generally acknowledged to include discomfort and/or pain along with disorders such as diarrhoea-predominant irritable bowel syndrome, constipation-predominant irritable bowel syndrome and alternater irritable bowel syndrome. (Gut, 1999; 45 (Suppl. II) II69-II77). In addition the Rome diagnostic criteria recognise that functional abdominal pain is distinct from functional bowel disorder and that functional abdominal pain includes functional abdominal pain syndrome and unspecified functional abdominal pain.

Gastroenterology 1999, 116, 1124-1131 discloses the role of NK₃ receptors on responses to colorectal distention in the rat via electrophysiological and behavioral studies. International Patent Application, Publication numbers 98/18762 and 00/21931 disclose certain NK₃ receptor antagonists stated to be useful for treating disorders involving NK3 receptors including irritable bowel syndrome (IBS) amongst a list of other disorders.

Certain selective NK₃ antagonists are disclosed in International Patent Application, Publication number 95/32948. These compounds are disclosed as having activity in the treatment of: pulmonary disorders (asthma, chronic obstructive pulmonary diseases -COPD-, airway hyperreactivity, cough), skin disorders and itch (for example, atopic dermatitis and cutaneous wheal and flare), neurogenic inflammation and CNS disorders (Parkinson's disease, movement disorders, anxiety and psychosis); and also the treatment of: convulsive disorders (for example epilepsy), renal disorders, urinary incontinence, ocular inflammation, inflammatory pain, eating disorders (food intake inhibition), allergic rhinitis, neurodegenerative disorders (for example Alzheimer's disease), psoriasis, Huntington's disease, and depression.

One particular compound disclosed in WO95/32948 is the compound of Example 85 therein being (S)-N-(α-ethylbenzyl)-3-hydroxy-2-phenylquinoline-4-carboxamide (Compound (I)).

It is now surprisingly indicated that Compound (I) has activity in the treatment of conditions characterised by altered bowel function and/or visceral pain. This includes, in particular, functional bowel disorders and functional abdominal pain. The functional bowel disorders include particular irritable bowel syndrome conditions, especially, diarrhoea-predominant irritable bowel syndrome, constipation-predominant irritable bowel syndrome and alternater irritable bowel syndrome. Compound (I) has been indicated to be particularly useful for treating diarrhoea-predominant irritable bowel syndrome.

Accordingly, the invention provides a method for the treatment and/or prophylaxis of conditions characterised by altered bowel function and/or visceral pain in humans or non-human mammals, which method comprises the administration of an effective, non-toxic and pharmaceutically acceptable amount of an NK₃ receptor antagonist, such as Compound (I), or a pharmaceutically acceptable derivative thereof, wherein the condition characterised by altered bowel function and/or visceral pain is selected from certain irritable bowel syndrome conditions, functional abdominal bloating, functional constipation, functional diarrhea, other bowel conditions and functional abdominal pain.

Suitably, the invention provides a method for the treatment and/or prophylaxis of conditions characterised by altered bowel function.

Suitably, the invention provides a method for the treatment and/or prophylaxis of conditions characterised by visceral pain.

Suitable conditions characterised by altered bowel function and/or visceral pain are selected from certain irritable bowel syndrome conditions, functional abdominal bloating, functional constipation, functional diarrhea and functional abdominal pain.

Particular irritable bowel syndrome conditions include diarrhoea-predominant irritable bowel syndrome, constipation-predominant irritable bowel syndrome and alternater irritable bowel syndrome.

A suitable irritable bowel syndrome condition is constipation-predominant irritable bowel syndrome.

A suitable irritable bowel syndrome condition is alternater irritable bowel syndrome.

A preferred irritable bowel syndrome condition is diarrhoea-predominant irritable bowel syndrome.

Suitably, functional abdominal pain includes functional abdominal pain syndrome and unspecified functional abdominal pain. Favourably, functional abdominal pain includes functional abdominal pain syndrome.

Suitable NK₃ antagonists include the compounds specifically and generically disclosed in International Patent Application, Publication number 95/32948 the contents of which are included herein by reference, as if the specific contents of WO 95/32948 were fully set forth herein.

A favoured NK₃ antagonist is a compound of formula (I):

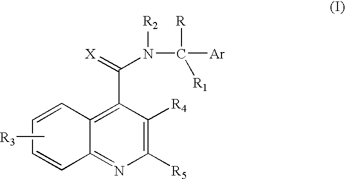

or a pharmaceutically acceptable solvate thereof, or a pharmaceutically acceptable salt thereof, wherein:

Ar is an optionally substituted phenyl, naphthyl or C₅₋₇ cycloalkdienyl group, or an optionally substituted single or fused ring heterocyclic group, having aromatic character, containing from 5 to 12 ring atoms and comprising up to four hetero-atoms in the or each ring selected from S, O, N;

R is linear or branched C₁₋₈ alkyl, C₃₋₇ cycloalkyl, C₄₋₇ cycloalkylalkyl, optionally substituted phenyl or phenyl C₁₋₆ alkyl, an optionally substituted five-membered heteroaromatic ring comprising up to four heteroatom selected from O and N, hydroxy C₁₋₆ alkyl, amino C₁₋₆ alkyl, C₁₋₆ alkylaminoalkyl, di C₁₋₆ alkylaminoalkyl, C₁₋₆ acylaminoalkyl, C₁₋₆ alkoxyalkyl, C₁₋₆ alkylcarbonyl, carboxy, C₁₋₆ alkoxyxcarbonyl, C₁₋₆ alkoxycarbonyl C₁₋₆ alkyl, aminocarbonyl, C₁₋₆ alkylaminocarbonyl, di C₁₋₆ alkylaminocarbonyl, halogeno C₁₋₆ alkyl; or is a group —(CH₂)ₚ— when cyclized onto Ar, where p is 2 or 3.

R₁ and R₂, which may be the same or different, are independently hydrogen or C₁₋₆ linear or branched alkyl, or together form a —(CH2)n— group in which n represents 3, 4, or 5; or R₁ together with R forms a group —(CH₂)_{q}—, in which q is 2, 3, 4 or 5;

R₃ and R₄, which may be the same or different, are independently hydrogen, C₁₋₆ linear or branched alkyl, C₁₋₆ alkenyl, aryl, C₁₋₆ alkoxy, hydroxy, halogen, nitro, cyano, carboxy, carboxamido, sulphonamido, C₁₋₆ alkoxycarbonyl, trifluoromethyl, acyloxy, phthalimido, amino, mono- and di-C₁₋₆ alkylamino, —O(CH₂)ᵣ—NT₂, in which r is 2, 3, or 4 and T is hydrogen or C₁₋₆ alkyl or it forms with the adjacent nitrogen a group

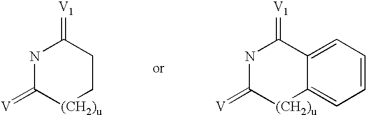

in which V and V₁ are independently hydrogen or oxygen and u is 0,1 or 2; —O(CH₂)ₛ—OW in which s is 2, 3, or 4 and W is hydrogen or C₁₋₆ alkyl; hydroxyalkyl, aminoalkyl, mono- or di-alkylaminoalkyl, acylamino, alkylsulphonylamino, aminoacylamino, mono- or di-alkylaminoacylamino; with up to four R₃ substituents being present in the quinoline nucleus; or R₄ is a group —(CH₂)ₜ— when cyclized onto R₅ as aryl, in which t is 1, 2, or 3;

R₅ is branched or linear C₁₋₆ alkyl, C₃₋₇ cycloalkyl, C₄₋₇ cycloalkylalkyl, optionally substituted aryl, or an optionally substituted single or fused ring heterocyclic group, having aromatic character, containing from 5 to 12 ring atoms and comprising up to four hetero-atoms in the or each ring selected from S, O, N;

X is O, S, or N—C≡N.

Suitable mammals are humans.

In particular the invention relates to a method for the treatment and/or prophylaxis of conditions characterised by altered bowel function and/or visceral pain in humans or non-human mammals, which method comprises the administration of an effective, non-toxic and pharmaceutically acceptable amount of a compound of formula (I), or a pharmaceutically acceptable derivative thereof.

Suitably, the invention relates to the treatment and/or prophylaxis of conditions characterised by altered bowel function in particular diarrhoea-predominant irritable bowel syndrome.

Preferably, the invention relates to the treatment and/or prophylaxis of diarrhoea.

Examples of Ar are phenyl, optionally substituted by hydroxy, halogen, C₁₋₆ alkoxy or C₁₋₆ alkyl. Examples of halogen are chlorine and fluorine, an example of C₁₋₆ alkoxy is methoxy and an example of C₁₋₆ alkyl is methyl.

Examples of Ar as a heterocyclic group are thienyl and pyridyl.

Examples of Ar as a C₅₋₇ cycloalkdienyl group is cyclohexadienyl.

Examples of R are as follows:

C₁₋₈ alkyl: methyl, ethyl, n-propyl, iso-propyl, n-butyl, heptyl;

phenyl C₁₋₆ alkyl: benzyl;

hydroxy C₁₋₆ alkyl: —CH₂OH, —CH₂CH₂OH, CH(Me)OH;

amino C₁₋₆ alkyl: —CH₂NH₂;

di C₁₋₆ alkylaminoalkyl: —CH₂NMe₂;

C₁₋₆ alkoxylalkyl: CH₂OMe;

C₁₋₆ alkylcarbonyl: COMe;

C₁₋₆ alkoxycarbonyl: COOMe;

C₁₋₆ alkoxycarbonyl C₁₋₆ alkyl: CH₂COOMe;

C₁₋₆ alkylaminocarbonyl: CONHMe;

di C₁₋₆ alkylaminocarbonyl: CONMe₂, CO(1-pyrrolidinyl);

halogen C₁₋₆ alkyl: trifluoromethyl;

—(CH₂)ₚ— when cyclized onto Ar:

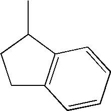

Example of R₁ and R₂ as C₁₋₆ alkyl is methyl; example of R₁ together with R forming a group —(CH₂)_{q}— is spirocyclopentane.

Examples of R₃ and R₄ are methyl, ethyl, n-propyl, n-butyl, methoxy, hydroxy, amino, chlorine, fluorine, bromine, acetyloxy, 2-(dimetylamino)ethoxy, 2-(phthalimido)ethoxy, aminoethoxy, 2-(1-pyrrolidinyl)ethoxy, phthalimido, dimethylaminopropoxy, dimethylaminoacetylamino, acetylamino, dimethylaminomethyl and phenyl.

Examples of R₅ are cyclohexyl, phenyl optionally substituted as defined for Ar above; examples of R₅ as a heterocyclic group are furyl, thienyl, pyrryl, thiazolyl, benzofuryl and pyridyl.

A preferred group of compounds of formula (I) are those in which:

Ar is phenyl, optionally substituted by C₁₋₆ alkyl or halogen; thienyl or a C₅₋₇ cycloalkdienyl group;

R is C₁₋₆ alkyl, C₁₋₆ alkoxycarbonyl, C₁₋₆ alkylcarbonyl, hydroxy C₁₋₆ alkyl;

R₁ and R₂ are each hydrogen or C₁₋₆ alkyl;

R₃ is hydrogen, hydroxy, halogen, C₁₋₆ alkoxy, C₁₋₆ alkyl;

R₄ is hydrogen, C₁₋₆ alkyl, C₁₋₆ alkoxy, hydroxy, amino, halogen, aminoalkoxy, mono- or di-alkylaminoalkoxy, mono- or di-alkylaminoalkyl, phthalimidoalkoxy, mono- or di-alkylaminoacylamino and acylamino;

R₅ is phenyl, thienyl, furyl, pyrryl and thiazolyl.

A further preferred group of compounds of formula (I) are those in which:

Ar is phenyl, 2-chlorophenyl, 2-thienyl or cyclohexadienyl;

R is methyl, ethyl, n-propyl, —COOMe, —COMe;

R₁ and R₂ are each hydrogen or methyl;

R₃ is hydrogen, methoxy, or hydroxy;

R₄ is hydrogen, methyl, ethyl, methoxy, hydroxy, amino, chlorine, bromine, dimethylaminoethoxy, 2-(phthalimido)ethoxy, aminoethoxy, 2-(1-pyrrolidinyl)ethoxy, dimethylaminopropoxy, dimethylaminoacetylamino, acetylamino, and dimethylaminomethyl.

R₅ is phenyl, 2-thienyl, 2-furyl, 2-pyrryl, 2-thiazolyl and 3-thienyl;

and X is oxygen.

A preferred sub-group of compounds within the scope of formula (I) above is of formula (Ia):

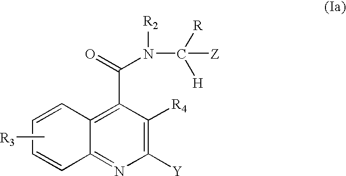

in which:

R, R₂, R₃ and R₄ are as defined in formula (I), and Y and Z, which may be the same or different, are each Ar as defined in formula (I).

A particularly preferred group of compounds of formula (I) are those of formula (Ib) in which the group R is oriented downward and H upward.

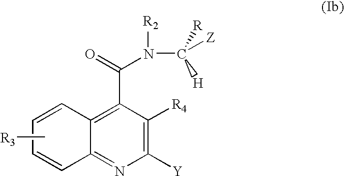

A most preferred compound of formula (I) is Compound (I).

The compounds of formula (I) or their derivatives such as salts or solvates are in pharmaceutically acceptable or substantially pure form. By pharmaceutically acceptable form is meant, inter alia, of a pharmaceutically acceptable level of purity excluding normal pharmaceutical additives such as diluents and carriers, and including no material considered toxic at normal dosage levels.

A substantially pure form will generally contain at least 50% (excluding normal pharmaceutical additives), preferably 75%, more preferably 90% and still more preferably 95% of the compound of formula (I) or its salt or solvate.

One preferred pharmaceutically acceptable form is the crystalline form, including such form in pharmaceutical composition. In the case of salts and solvates the additional ionic and solvent moieties must also be non-toxic.

As indicated above suitable pharmaceutically acceptable derivatives include pharmaceutically acceptable salts and/or solvates.

Examples of pharmaceutically acceptable salts of a compound of formula (I) include the acid addition salts with the conventional pharmaceutical acids, for example maleic, hydrochloric, hydrobromic, phosphoric, acetic, fumaric, salicylic, citric, lactic, mandelic, tartaric, succinic, benzoic, ascorbic, and methanesulphonic.

Examples of pharmaceutically acceptable solvates of a compound of formula (I) include hydrates.

The compounds of formula (I) may have at least one asymmetric centre and therefore may exist in more than one stereoisomeric form. The treatment of the invention extends to all such forms and to mixtures thereof, including racemates.

Other NK₃ antagonists are those disclosed in published patent applications WO99/36424, WO00/39114, WO95/28931, WO96/05203, EP776893, WO98/54191, EP673928, WO94/26735 and WO97/10229. The contents of the above mentioned patent publications are incorporated herein as if each individual publication were specifically and individually incorporated by reference herein as though fully set forth. A particular NK₃ antagonist is ([[dichlorophenyl)(trimethoxybenzoy)morpholinyl]ethyl]spiro[benzo(c)thiophenepiperidine] oxide. A particular NK₃ antagonist is R113281. A particular NK₃ antagonist is N10A. A particular NK₃ antagonist is N5A1. A particular NK₃ antagonist is SR-142801. A particular NK₃ antagonist is SSR-146977. A particular NK₃ antagonist is Cam-2425. A particular NK₃ antagonist is MDL-105212.

The NK₃ antagonist is prepared according to known methods for the particular compound chosen, for example compounds of formula (I) and Compound (I) are prepared according to methods disclosed in WO95/32948 or WO99/14196. Other NK₃ antagonists such as R113281, N10A, N5A1, SR-142801, SSR-146977, Cam-2425 and MDL-105212 are prepared, as appropriate, according to published methods for example those disclosed in WO99/36424, WO00/39114, WO95/28931, WO96/05203, EP776893, WO98/54191, EP673928, WO94/26735 and WO97/10229.

The compounds of the method of the invention such as Compound (I), may be converted into their pharmaceutically acceptable acid addition salts by reaction with the appropriate organic or mineral acids.

Solvates of the compounds of the method of the invention such as Compound (I), may be formed by crystallization or recrystallization from the appropriate solvent. For example, hydrates may be formed by crystallization or recrystallization from aqueous solutions, or solutions in organic solvents containing water.

Pharmaceutically acceptable derivatives of other NK₃ antagonists such as R113281, N10A, N5A1, SR-142801, SSR-146977, Cam-2425 and MDL-105212 include, as appropriate, those disclosed in WO99/36424, WO00/39114, WO95/28931, WO96/05203, EP776893, WO98/54191, EP673928, WO94/26735 and WO97/10229.

The activity of the compounds of the method of the invention such as Compound (I), as NK₃ receptor antagonists are assessed in standard tests indicates that they are of potential therapeutic utility in the treatment of certain clinical conditions characterized by overstimulation of the tachykinin receptors, in particular the conditions disclosed herein. The relevant tests include those disclosed herein.

When used herein “conditions characterised by altered bowel function” includes diarrhoea-predominant irritable bowel syndrome, constipation-predominant irritable bowel syndrome, alternater irritable bowel syndrome, functional abdominal bloating, functional constipation and functional diarrhea

In particular conditions characterised by altered bowel function includes diarrhoea-predominant irritable bowel syndrome.

In particular conditions characterised by altered bowel function includes constipation-predominant irritable bowel syndrome.

In particular conditions characterised by altered bowel function includes alternater irritable bowel syndrome

In particular conditions characterised by altered bowel function includes other bowel conditions.

When used herein “other bowel conditions” includes conditions presenting with symptoms such as abdominal pain, urgency, bloating, incomplete evacuation and straining, especially urgency, bloating, incomplete evacuation and straining.

The present invention also provides a method for the treatment and/or prophylaxis of conditions characterised by altered bowel function and/or visceral pain, in humans or non-human mammals, which method comprises the administration of an effective, non-toxic and pharmaceutically acceptable amount of an NK₃ antagonist, such as Compound (I), or a pharmaceutically acceptable derivative thereof

The present invention also provides an NK₃ antagonist, such as Compound (I) or a pharmaceutically acceptable derivative thereof, for use in the treatment and/or prophylaxis of conditions characterised by altered bowel function and/or visceral pain.

There is also provided the use of an NK₃ antagonist, such as Compound (I) or a pharmaceutically acceptable derivative thereof, in the manufacture of a medicament for the treatment and/or prophylaxis of conditions characterised by altered bowel function and/or visceral pain. Suitably wherein the condition characterised by altered bowel function and/or visceral pain is selected from certain irritable bowel syndrome conditions, functional abdominal bloating, functional constipation, functional diarrhea, other bowel conditions and functional abdominal pain.

The present invention further provides a pharmaceutical composition comprising an NK₃ antagonist, such as a compound of formula (I), or a pharmaceutically acceptable salt thereof or a or pharmaceutically acceptable solvate thereof, and a pharmaceutically acceptable carrier therefor, for the treatment and/or prophylaxis of conditions characterised by altered bowel function and/or visceral pain suitably wherein the condition characterised by altered bowel function and/or visceral pain is selected from certain irritable bowel syndrome conditions, functional abdominal bloating, functional constipation, functional diarrhea, other bowel conditions and functional abdominal pain.

Such a medicament, and a composition of this invention, may be prepared by admixture of a compound of the invention with an appropriate carrier. It may contain a diluent, binder, filler, disintegrant, flavouring agent, colouring agent, lubricant or preservative in conventional manner.

These conventional excipients may be employed for example as in the preparation of compositions of known agents for treating the conditions.

Preferably, a pharmaceutical composition of the invention is in unit dosage form and in a form adapted for use in the medical or veterinarial fields. For example, such preparations may be in a pack form accompanied by written or printed instructions for use as an agent in the treatment of the conditions.

The suitable dosage range for the compounds of the invention depends on the compound to be employed and on the condition of the patient. It will also depend, inter alia, upon the relation of potency to absorbability and the frequency and route of administration.

The compound or composition of the invention may be formulated for administration by any route, and is preferably in unit dosage form or in a form that a human patient may self-administer it in a single dosage. Advantageously, the composition is suitable for oral, rectal, topical, parenteral, intravenious or intramuscular administration. Preparations may be designed to give slow release of the active ingredient.

Compositions may, for example, be in the form of tablets, capsules, sachets, vials, powders, granules, lozenges, reconstitutable powders, or liquid preparations, for example solutions or suspensions, or suppositories.

The compositions, for example those suitable for oral administration, may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinyl-pyrrolidone, sodium starch glycollate or microcrystalline cellulose; or pharmaceutically acceptable setting agents such as sodium lauryl sulphate.

Solid compositions may be obtained by conventional methods of blending, filling, tabletting or the like. Repeated blending operations may be used to distribute the active agent throughout those compositions employing large quantities of fillers. When the composition is in the form of a tablet, powder, or lozenge, any carrier suitable for formulating solid pharmaceutical compositions may be used, examples being magnesium stearate, starch, glucose, lactose, sucrose, rice flour and chalk. Tablets may be coated according to methods well known in normal pharmaceutical practice, in particular with an enteric coating. The composition may also be in the form of an ingestible capsule, for example of gelatin containing the compound, if desired with a carrier or other excipients.

Compositions for oral administration as liquids may be in the form of, for example, emulsions, syrups, or elixirs, or may be presented as a dry product for reconstitution with water or other suitable vehicle before use. Such liquid compositions may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; aqueous or non-aqueous vehicles, which include edible oils, for example almond oil, fractionated coconut oil, oily esters, for example esters of glycerine, or propylene glycol, or ethyl alcohol, glycerine, water or normal saline; preservatives, for example methyl or propyl p-hydroxybenzoate or sorbic acid; and if desired conventional flavouring or colouring agents.

The active compounds of this invention may also be administered by a non-oral route. In accordance with routine pharmaceutical procedure, the compositions may be formulated, for example for rectal administration as a suppository. They may also be formulated for presentation in an injectable form in an aqueous or non-aqueous solution, suspension or emulsion in a pharmaceutically acceptable liquid, e.g. sterile pyrogen-free water or a parenterally acceptable oil or a mixture of liquids. The liquid may contain bacteriostatic agents, anti-oxidants or other preservatives, buffers or solutes to render the solution isotonic with the blood, thickening agents, suspending agents or other pharmaceutically acceptable additives. Such forms will be presented in unit dose form such as ampoules or disposable injection devices or in multi-dose forms such as a bottle from which the appropriate dose may be withdrawn or a solid form or concentrate which can be used to prepare an injectable formulation.

The active compounds of this invention may also be administered by inhalation, via the nasal or oral routes. Such administration can be carried out with a spray formulation comprising a compound of the invention and a suitable carrier, optionally suspended in, for example, a hydrocarbon propellant.

Preferred spray formulations comprise micronised compound particles in combination with a surfactant, solvent or a dispersing agent to prevent the sedimentation of suspended particles. Preferably, the compound particle size is from about 2 to 10 microns.

A further mode of administration of the active compounds of this invention comprises transdermal delivery utilising a skin-patch formulation. A preferred formulation comprises a compound of the invention dispersed in a pressure sensitive adhesive which adheres to the skin, thereby permitting the compound to diffuse from the adhesive through the skin for delivery to the patient. For a constant rate of percutaneous absorption, pressure sensitive adhesives known in the art such as natural rubber or silicone can be used.

As mentioned above, the effective dose of compound depends on the particular compound employed, the condition of the patient and on the frequency and route of administration. A unit dose will generally contain from 20 to 1000 mg and preferably will contain from 30 to 500 mg, in particular 50, 100, 150, 200, 250, 300, 350, 400, 450, or 500 mg. The composition may be administered once or more times a day for example 2, 3 or 4 times daily, and the total daily dose for a 70 kg adult will normally be in the range 100 to 3000 mg. Alternatively the unit dose will contain from 2 to 20 mg of active ingredient and be administered in multiples, if desired, to give the preceding daily dose.

Dosages and formulations of particular NK₃ antagonists include those disclosed in the above mentioned patent applications.

No unacceptable toxicological effects are expected with the compounds of the method of the invention when administered in accordance with the invention.

The activity of the compounds of the present invention, as NK₃ ligands, is determined by their ability to inhibit the binding of the radiolabelled NK₃ ligands, [¹²⁵I]-[Me-Phe⁷]-NKB or [³H]-Senktide, to guinea-pig and human NK₃ receptors (Renzetti et al, 1991, Neuropeptide, 18, 104-114; Buell et al, 1992, FEBS, 299(1), 90-95; Chung et al, 1994, Biochem. Biophys. Res. Commun., 198(3), 967-972). The binding assays utilized allow the determination of the concentration of the individual compound required to reduce by 50% the [¹²⁵I]-[Me-Phe⁷]-NKB and [³H]-Senktide specific binding to NK₃ receptor in equilibrium conditions (IC50). Binding assays provide for each compound tested a mean IC₅₀ value of 2-5 separate experiments performed in duplicate or triplicate. The most potent compounds of the present invention show IC₅₀ values in the range 1-1000 nM; in particular, in guinea-pig cortex membranes by displacement of [³H]-Senktide, the compounds of the Examples 22, 47, 48, and 85 display Kᵢs (nM) of 5.6, 8.8, 12.0 and 4.8 respectively (n=3). The NK₃-antagonist activity of the compounds of the present invention is determined by their ability to inhibit senktide-induced contraction of the guinea-pig ileum (Maggi et al, 1990, Br. J. Pharmacol., 101, 996-1000) and rabbit isolated iris sphincter muscle (Hall et al., 1991, Eur. J. Pharmacol., 199, 9-14) and human NK₃ receptors-mediated Ca⁺⁺ mobilization (Mochizuki et al, 1994, J. Biol. Chem., 269, 9651-9658). Guinea-pig and rabbit in-vitro functional assays provide for each compound tested a mean K_{B} value of 3-8 separate experiments, where K_{B} is the concentration of the individual compound required to produce a 2-fold rightward shift in the concentration-response curve of senktide. Human receptor functional assay allows the determination of the concentration of the individual compound required to reduce by 50% (IC₅₀ values) the Ca⁺⁺ mobilization induced by the agonist NKB. In this assay, the compounds of the present invention behave as antagonists.

For the avoidance of doubt, all publications, including but not limited to patents and patent applications, cited in this specification are herein incorporated by reference as if each individual publication were specifically and individually indicated to be incorporated by reference herein as though fully set forth.

The therapeutic potential of the compounds of the present invention in treating the conditions mentioned above can be assessed using rodent disease models.

The invention is illustrated, but not limited by, the following experimental data.

### FIGURES

The following is a brief description of the figures referred to below:

FIG. 1: shows inhibition by Compound (I) of the ascending excitory reflex;

FIG. 2: shows the lack of significant effect of Compound (I) on small intestinal transit in normal non-sensitised animals;

FIG. 3: shows the effect of Compound (I) on small intestinal transit in egg-albumen sensitisation model; and

FIG. 4: shows the effect of Compound (I) on rat colorectal sensitivity and tone.

### EXPERIMENTAL RESULTS

Inability of Compound (I) to Affect Normal Gastrointestinal Motility

Compound (I) at oral doses of 5, 15 and 50 mg/kg produced no marked or statistically significant effects on rat gastric emptying, as determined using the phenol red method. The high doses used in this study are similar to those that exerted intestinal antinociceptive activity (see below) and are consistent with the low affinity of Compound (I) for the rat NK-3 receptor, relative to its higher affinity for the human or guinea-pig variants of this receptor. By contrast, the reference standard, morphine sulfate administered orally at 20 mg/kg produced a marked and statistically significant decrease in gastric emptying (see Table 1). This effect was expected and demonstrated the validity of the test system.

TABLE 1
Proportion of gastric emptying in rats after administration of
Compound (I), morphine sulfate, phenol red and vehicle only

| | | | % GASTRIC |
|---|---|---|---|
| GROUP | ORAL TREATMENT | DOSE (mg/kg) | EMPTYING |
| 1 | Phenol red only | — | 0.0 |
| 2 | Vehicle | — | 60.6 |
| 3 | Compound (I) | 5 | 55.2 |
| 4 | Compound (I) | 15 | 50.7 |
| 5 | Compound (I) | 50 | 62.4 |
| 6 | Morphine sulfate | 20 | 30.8** |

Significance of difference from vehicle-treated group:
**p < 0.01

Oral administration of Compound (I) at doses of 5, 15 and 50 mg/kg also produced no marked or statistically significant effect on gastrointestinal motility in the rat, as determined using the charcoal meal test. Thus, the distance travelled by the charcoal meal in each Compound (I)-treated group was similar to the vehicle-treated group at each tested dose. By contrast, morphine sulfate at an oral dose of 10 mg/kg produced a marked and statistically significant decrease in gastrointestinal motility compared with the vehicle-treated group (see Table 2). This effect was expected and demonstrated the validity of the test system.

TABLE 2
Effect of Compound (I), morphine sulfate, and vehicle on
gastrointestinal motility in rats, as determined by the
charcoal meal test

| | | | GROUP | |
|---|---|---|---|---|
| | | | MEAN DISTANCE | |
| | | | TRAVELLED | % CHANGE |
| | | | BY CHARCOAL | FROM |
| | | | AS % OF | VEHICLE- |
| | ORAL | DOSE | TOTAL GUT | TREATED |
| GROUP | TREATMENT | (mg/kg) | LENGTH ± SD | ANIMALS |
| 1 | Vehicle | — | 53.0 ± 7.6 | — |
| 2 | Compound (I) | 5 | 46.9 ± 5.3 | −11.5 |
| 3 | Compound (I) | 15 | 52.1 ± 7.1 | −1.7 |
| 4 | Compound (I) | 50 | 50.4 ± 8.6 | −4.9 |
| 5 | Morphine | 10 | 37.4 ± 12.4 | −29.4 |
| | sulfate | | | |

SD = standard deviation
Statistical significance of difference from vehicle-treated group:
**p < 0.01

Inhibition by Compound (I) of Slow EPSP in Guinea-Pig Enteric Neurones

Four neurons were analyzed with Compound (I) to determine its effects on the slowEPSP. The slowEPSP was elicited by high frequency (20 Hz) electrical stimulation for 1 s, applied to a circumferential internodal strand connecting to the ganglion. A baseline slowEPSP was obtained and once it subsided, Compound (I) at a concentration of 0.1 microM was perfused through the preparation. It was perfused continuously and the slowEPSP was tested at various intervals. In all four neurons there was a decrease of peak amplitude of depolarisation of over 50% when compared with baseline, once the drug had circulated for at least 30 minutes. There was 100% blockade in one neuron and it even revealed an inhibitory post-synaptic potential (IPSP) on stimulation. The other three neurons showed a blockade, based on the amplitude of depolarisation, of 60-80%. Two of the neurons were filled with biocytin and reacted with streptavidin and Texas Red revealing a Dogiel type II morphology on fluorescence microscopy.

Inhibition by Compound (I) of Stretch-Induced Reflex Activity in Guinea-Pig Isolated Colon

For reflex studies, 5-7 cm lengths of colon were suspended in warmed, oxygenated physiological saline, so that an 8 mm metal bar could be inserted into the lumen and connected, via a thread passing through the intestinal wall, to a pulley system which allowed different weights to pull on the bar and distend the intestine. Force transducers were attached to the wall of the colon via small wire hooks, 5 mm either side of the distending bar. Colonic wall distension using 6, 12 or 20 g weights evoked rhythmic contractions oral to the distension and a small relaxation on the anal side. Application of 12 and 20 g weights evoked similar patterns of activity and although the small relaxation on the anal side of the distension seemed more apparent, the oral excitatory response was not different from the effects of the 6 g weight. Compound (I) 250 nM, applied for 30-45 min, reduced the effects of distension in an approximately distension-dependent manner, with the effects being greater at the higher levels of distension (see Figure below). These data suggest that NK₃ receptor antagonism by Compound (I) reduces ascending and descending reflexes evoked by distension. Since the effects of Compound (I) on enteric reflexes were more apparent at the higher distension weights, NK₃ receptors and hence, the slow EPSP component of IPAN activity, may be involved to a greater extent in reflexes evoked using intensive and possibly pathological stimuli.

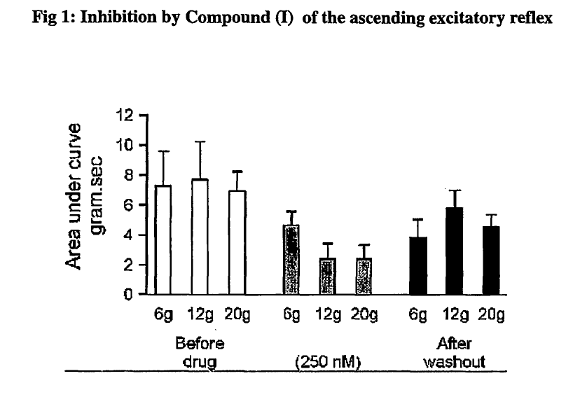

The date are represented in FIG. 1 below and show the changes in the ascending excitatory reflex, measured as area under the curve in g.sec., induced by 6, 12 or 20 g distension, in the absence and presence of Compound (I) and after washout.

Inhibition by Compound (I) of the Egg-Albumin Sensitized Increase in Rat Small Intestinal Transit

Summary and Conclusions

For the first time, NK3 receptor antagonism by Compound (I) 15 mg/ kg po., has been shown to inhibit the increase in rat small intestinal transit time evoked by an egg-albumin challenge. These data strongly support an involvement of the NK3 receptor in pathological (rather than physiological) changes in intestinal function.

Objectives

Increased rat small intestinal transit induced by sensitization to an egg-albumin immune challenge has previously been used to demonstrate the ability of the 5-HT3 receptor antagonist, alosetron, to normalise disturbed patterns of intestinal transit (Clayton N M. Sargent R. Butler A. Gale J. Maxwell M P. Hunt A A. Barrett V J. Cambridge D. Bountra C. Humphrey P P. The pharmacological properties of the novel selective 5-HT3 receptor antagonist, alosetron, and its effects on normal and perturbed small intestinal transit in the fasted rat. Neurogastroenterology & Motility. 11(3):207-17, 1999 June). Given both the significance of the data with alosetron and the potential interaction between such challenges within the gut and the NK3 receptor (Gay J, Fioramonti J, Garcia-Villar R, Edmonds-Alt X, Bueno L (1999) Gut, 44,497-503.), the effects of Compound (I) were studied in this model.

Results

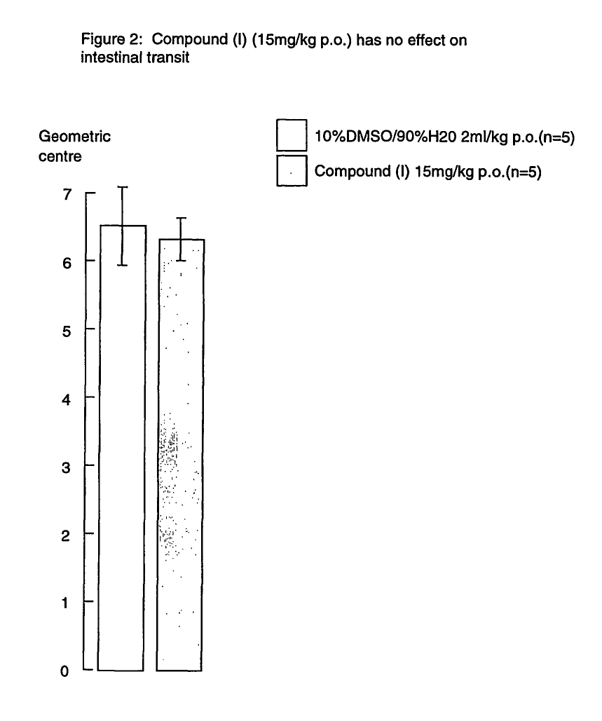

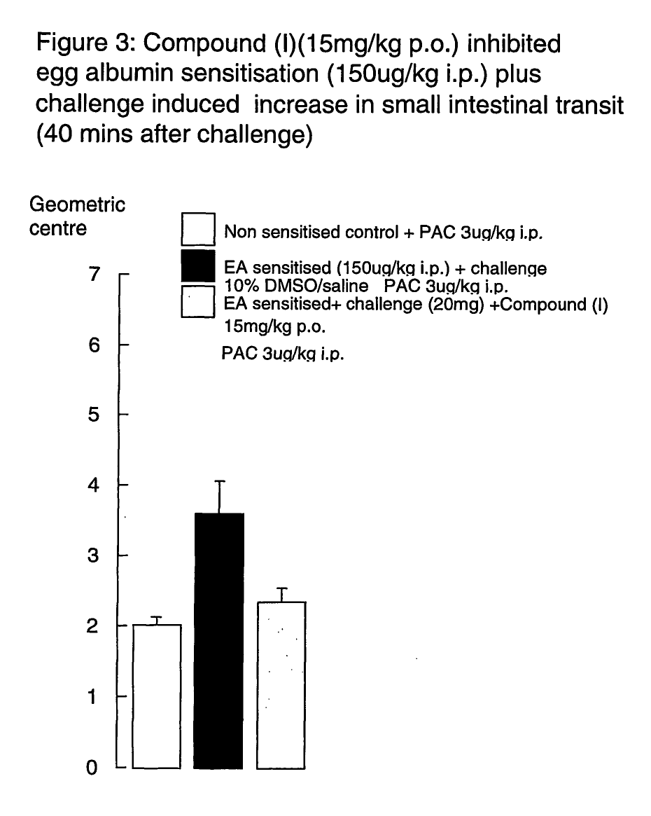

Compound (I) 15 mg/ kg po did not significantly affect small intestinal transit in normal, non-sensitised animals (see FIG. 2 below). However, in the egg-albumin sensitisation model, the increased transit seen after egg-albumin sensitisation was greatly inhibited by pre-treatment with compound (I) 15 mg/kg po.,(see FIG. 3).

Discussion

These data demonstrate, for the first time with Compound (I), that in contrast to the lack of involvement of the NK3 receptor in normal patterns of intestinal motility, this receptor may have significant function in mediating disturbed patterns of motility. Further, these data and the use of this model now makes it possible to correlate the significances of those data obtained in vitro (demonstrated involvement of NK3 receptors in IPAN slow EPSP activity; demonstrated ability of NK3 receptor activation to inhibit non-cholinergic excitatory nerve activity in human isolated colon) with in vivo intestinal pathology.

Inhibition by Compound (I) of Intestinal Nociception

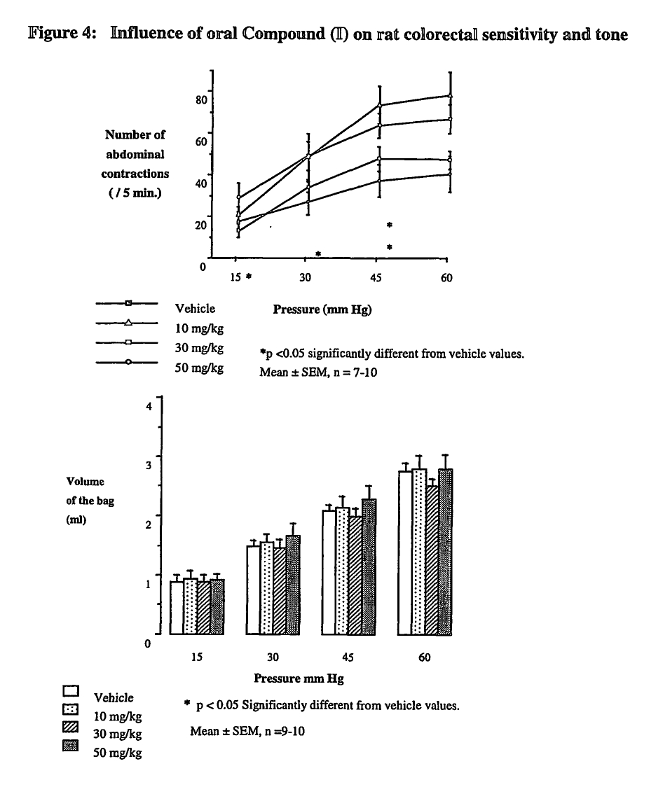

In conscious rats, Compound (I) 50 mg/kg, po had a significant antinociceptive effect during distension of the colo-rectal area at 30, 45 and 60 mmHg distension pressures, without affecting colo-rectal tone (see FIG. 4 below). Compound (I) 30 mg/kg, po acted similarly, but the effects were statistically significant only at the highest distension pressure. The lowest dose of Compound (I) tested (10 mg/kg, po) was without anti-nociceptive activity.

These data indicate that selective NK-3 receptor antagonism can reduce intestinal sensitivity to noxious distension.

1. A method for the treatment and/or prophylaxis of conditions characterised by altered bowel function and/or visceral pain in humans or non-human mammals, which method comprises the administration of an effective, non-toxic and pharmaceutically acceptable amount of an NK₃ receptor antagonist, wherein the condition characterised by altered bowel function and/or visceral pain is selected from certain irritable bowel syndrome conditions, functional abdominal bloating, functional constipation, functional diarrhea, other bowel conditions and functional abdominal pain.

2. A method according to claim 1, for the treatment and/or prophylaxis of conditions characterised by altered bowel function.

3. A method according to claim 1, wherein the irritable bowel syndrome condition is diarrhoea-predominant irritable bowel syndrome.

4. A method for the treatment and/or prophylaxis of conditions characterised by altered bowel function and/or visceral pain in humans or non-human mammals, which method comprises the administration of an effective, non-toxic and pharmaceutically acceptable amount of a compound of formula (I): 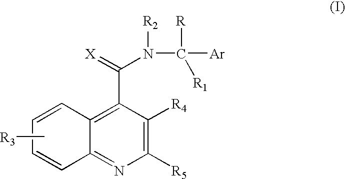
or a pharmaceutically acceptable solvate thereof, or a pharmaceutically acceptable salt thereof, wherein:
Ar is an optionally substituted phenyl, naphthyl or C₅₋₇ cycloalkdienyl group, or an optionally substituted single or fused ring heterocyclic group, having aromatic character, containing from 5 to 12 ring atoms and comprising up to four hetero-atoms in the or each ring selected from S, O, N;
R is linear or branched C₁₋₈ alkyl, C₃₋₇ cycloalkyl, C₄₋₇ cycloalkylalkyl, optionally substituted phenyl or phenyl C₁₋₆ alkyl, an optionally substituted five-membered heteroaromatic ring comprising up to four heteroatom selected from O and N, hydroxy C₁₋₆ alkyl, amino C₁₋₆ alkyl, C₁₋₆ alkylaminoalkyl, di C₁₋₆ alkylaminoalkyl, C₁₋₆ acylaminoalkyl, C₁₋₆ alkoxyalkyl, C₁₋₆ alkylcarbonyl, carboxy, C₁₋₆ alkoxyxcarbonyl, C₁₋₆ alkoxycarbonyl C₁₋₆ alkyl, aminocarbonyl, C₁₋₆ alkylaminocarbonyl, di C₁₋₆ alkylaminocarbonyl, halogeno C₁₋₆ alkyl; or is a group —(CH₂)ₚ— when cyclized onto Ar, where p is 2 or 3.
R₁ and R₂, which may be the same or different, are independently hydrogen or C₁₋₆ linear or branched alkyl, or together form a —(CH2)n— group in which n represents 3, 4, or 5; or R₁ together with R forms a group —(CH₂)_{q}—, in which q is 2, 3, 4 or 5;
R₃ and R₄, which may be the same or different, are independently hydrogen, C₁₋₆ linear or branched alkyl, C₁₋₆ alkenyl, aryl, C₁₋₆ alkoxy, hydroxy, halogen, nitro, cyano, carboxy, carboxamido, sulphonamido, C₁₋₆ alkoxycarbonyl, trifluoromethyl, acyloxy, phthalimido, amino, mono- and di-C₁₋₆ alkylamino, —O(CH₂)ᵣ—NT₂, in which r is 2, 3, or 4 and T is hydrogen or C₁₋₆ alkyl or it forms with the adjacent nitrogen a group 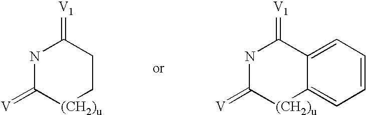
in which V and V₁ are independently hydrogen or oxygen and u is 0,1 or 2; —O(CH₂)ₛ—OW in which s is 2, 3, or 4 and W is hydrogen or C₁₋₆ alkyl; hydroxyalkyl, aminoalkyl, mono-or di-alkylaminoalkyl, acylamino, alkylsulphonylamino, aminoacylamino, mono- or di-alkylaminoacylamino; with up to four R₃ substituents being present in the quinoline nucleus; or R₄ is a group —(CH₂)ₜ— when cyclized onto R₅ as aryl, in which t is 1, 2, or 3;
R₅ is branched or linear C₁₋₆ alkyl, C₃₋₇ cycloalkyl, C₄₋₇ cycloalkylalkyl, optionally substituted aryl, or an optionally substituted single or fused ring heterocyclic group, having aromatic character, containing from 5 to 12 ring atoms and comprising up to four hetero-atoms in the or each ring selected from S, O, N;
X is O, S, or N—C≡N.

5. A method according to claim 4, for the treatment and/or prophylaxis of conditions characterised by altered bowel function.

6. A method according to claim 4, wherein the irritable bowel syndrome condition is diarrhoea-predominant irritable bowel syndrome.

7. A method according to claim 4, wherein the compound of formula (I) is (S)-N-(α-ethylbenzyl)-3-hydroxy-2-phenylquinoline-4-carboxamide (Compound (I)).

8. A method for the treatment and/or prophylaxis of conditions characterised by altered bowel function and/or visceral pain in humans or non-human mammals, which method comprises the administration of an effective, non-toxic and pharmaceutically acceptable amount of an NK₃ antagonist selected from the list consisting of: ([[dichlorophenyl)(trimethoxybenzoy)morpholinyl]ethyl]spiro[benzo(c)thiophenepiperidine]oxide, R113281, N10A, N5A1, SR-142801, SSR-146977, Cam-2425 and MDL-105212. or, as appropriate, a pharmaceutically acceptable derivative thereof

9. A method according to claim 8, for the treatment and/or prophylaxis of conditions characterised by altered bowel function.

10. A method according to claim 8, wherein the irritable bowel syndrome condition is diarrhoea-predominant irritable bowel syndrome.
